(19) United States
(12) Patent Application Publication
(10) Pub. No.: US 2003/0018333 A1
(43) Pub. Date: Jan. 23, 2003

(54) Device and method to protect wounds and orthopedic pins from external contact

(76) Inventor: Fritz C. Kokesh, Cambridge, MA (US)

Correspondence Address: Dike, Bronstein, Roberts & Cushman, Intellectual Property Practice Group, EDWARDS & ANGELL, LLP, P.O. Box 9169, Boston, MA, 02209, US

(21) Appl. No.: 09/910,196

(22) Filed: Jul. 20, 2001

(51) Int. Cl. A61B 17/56

(52) U.S. Cl. 606/53

(57) ABSTRACT

The present invention discloses a device, system, and method for protecting orthopedic pins used to fixate fractured bones that protrude through a patient's skin, post-operative dressing or cast from multidirectional contact with external objects. One aspect of the device comprises a rigid upper body that is configured and arranged to encompass and protect the orthopedic pins and a conformable lower body, e.g., a plurality of flexible legs, for removably attaching the rigid upper body to a surface surrounding the protruding pins. One aspect of the system comprises the device in combination with an attaching device, which removably attaches the conformable lower body to the surface surrounding the orthopedic pins. One aspect of the method includes the steps of configuring and arranging a rigid upper body to encompass and protect the orthopedic pins and removably attaching the conformable lower body to a surface surrounding the orthopedic pins.

### FIELD OF THE INVENTION

The present invention relates to orthopedic surgical devices. More precisely, the invention relates to devices and methods for protecting surficial wounds and/or surgical pins that have been inserted to fixate bone segments and that protrude through a patient's skin, post-operative dressing, or cast.

### DESCRIPTION OF THE RELATED ART

Orthopedic surgical techniques, particularly those intended to align segments of broken bones, often involve the insertion of one or more surgical pins, which are intended to fixate fractured bone segments in proper alignment until the bone segments have healed properly. In many instances, these surgical pins protrude through the epidermis, i.e., the skin, to facilitate their removal after the bone has mended. Moreover, in many instances, these pins further protrude through a post-operative dressing or cast so that the wound in the vicinity of the protruding pins can be inspected and cleaned regularly.

Problems associated with protruding orthopedic pins include, inter alia, the pain and/or injury to the patient, which can result from contact of the protruding pins with clothing and/or other external objects, and, further, the protruding pins, which oftentimes have sharp ends, can injure people or damage property, e.g., by scratching. Accordingly, protruding orthopedic pins are often shunted, bent down, and/or provided with an end cap made of rubber or plastic. However, these methods do not remedy or completely alleviate the above-described problems. Indeed, these measures, especially the end cap, merely minimize external injury to others and/or damage to property. They do not protect against the pain or further injury to the patient that might result from external contact with and the application of multidirectional, external loads or forces on the pins.

U.S. Pat. No. 5,499,982 to Adamson discloses a device to protect the end of a surgical pin protruding from an injured digit, e.g., a finger or toe, from a blow directed along the axis of the pin. The device comprises a flare-shaped protective cavity that is open at both ends in which the protruding pin is disposed. The size of the protective device is such that the protruding pin does not contact the inner wall of the cavity. Furthermore, the protective device can be held in place on the tip of a patient's finger or toe using two or more retaining strips or by allowing the device to partially extend over the tip of a patient's finger or toe and applying tape. A method of protecting the end of a surgical pin protruding from a patient's finger or toe using the above-described device is disclosed, further, in U.S. Pat. No. 5,752,952 to Adamson.

Problems with the Adamson inventions include (i) that they only protect against blows or forces applied in an axial direction; (ii) they are directed expressly to fingers and toes; and (iii) they are difficult to attach securely.

### SUMMARY OF THE INVENTION

Accordingly, it would be desirable to produce a removable device, system, and method for protecting protruding orthopedic pins to minimize pain and injury to the patient due to multidirectional contact with an external object and for protecting those objects and persons that come into contact with the patient during the period needed for the injury to heal and/or while the surgical pins are in place.

Therefore, it is an object of the present invention to produce a device, system, and method for encompassing one or more surgical pins, which protrude through a patient's skin, post-operative dressing or cast, to protect the surgical pins from multidirectional contact with external objects.

It is yet another object of the present invention to produce a device, system, and method for encompassing one or more surgical pins, which protrude through a patient's skin, post-operative dressing or cast, to protect external objects from damage or injury resulting from contact with the exposed surgical pins.

It is a further object of the present invention to produce a device, system, and method for encompassing one or more surgical pins, which protrude through a patient's skin, post-operative dressing or cast, that can be applied and removed quickly and easily in order to allow inspection and cleaning of the wound in the vicinity of the protruding pins.

It is also an object of present invention to produce a device, system, and method for encompassing one or more surgical pins, which protrude through a patient's skin, post-operative dressing or cast, whereby the device conceals the wounds and/or pins from view to protect the patient's privacy.

It is still another object of the present invention to produce a device, system, and method for encompassing one or more surgical pins, which protrude through a patient's skin, post-operative dressing or cast, that also allows air to circulate about the pins and wound.

It is yet another object of the present invention to produce a device, system, and method for encompassing one or more surgical pins, which protrude through a patient's skin, post-operative dressing or cast, that allows patient's to dress in regular clothing without concern of contacting the surgical pins while dressing or undressing.

These and further objects are obtained by a device for protecting surgical pins, which protrude through a patient's skin, post-operative dressing or cast, the device comprising a rigid upper body, having a shape that includes an inner cavity, an upper surface, and an outer wall, for encompassing the surgical pins to protect them from multidirectional external loads or forces; and a conformable lower body having a plurality of flexible legs, or tabs, for removably attaching the device to a surface surrounding the surgical pins.

In another aspect of the present invention, there is disclosed a system for protecting surgical pins, which protrude through a patient's skin, postoperative dressing or cast, the system comprising (i) a removable protecting device for encompassing one or more surgical pins, which includes a rigid upper body to protect the pins and a conformable lower body having a plurality of flexible legs for attaching the device to a surface surrounding the pins; and (ii) an attaching device for removably attaching the protecting device to a surface surrounding the surgical pins.

In yet another aspect of the present invention, there is disclosed a method of protecting one or more surgical pins, which protrude through a patient's skin, post-operative dressing or cast, the method comprising the steps of (i) encompassing the surgical pins in a rigid upper body of a shielding device, and (ii) removably attaching a conformable lower body of the shielding device to a surface surrounding the surgical pins using an attaching device.

### BRIEF DESCRIPTION OF THE DRAWINGS

For a fuller understanding of the nature and desired objects of the present invention, reference is made to the following detailed description taken in conjunction with the accompanying figures wherein like reference character denote corresponding parts throughout the several views and wherein:

FIG. 1a is an illustrative embodiment of prior art methods of protecting orthopedic pins protruding through a cast by shunting the pins;

FIG. 1b is an illustrative embodiment of prior art methods of protecting orthopedic pins protruding through a cast using an end cap made of rubber or plastic;

FIG. 2a is a first isometric view of an illustrative embodiment of one aspect of the protective device;

FIG. 2b is a second isometric view of an illustrative embodiment of another aspect of the protective device; and

FIG. 3 is an illustrative embodiment of one aspect of the protective system being applied to a cast.

### DETAILED DESCRIPTION OF THE INVENTION AND ITS PREFERRED EMBODIMENTS

In the medical world, there exist a variety of orthopedic pins and fasteners (collectively “pins”) that are used, primarily, to properly fixate, or arrest, bones or bone portions to further the natural healing of the fractured bone. The pins can be, for example, of a screw-, anchor-, and/or nail-type and, moreover, can be used singly or in groups. Once the fractured bone has healed, the pins must be removed. To facilitate removal of the pins, surgeons customarily allow the pins to extend through the patient's skin or post-operative dressing. Moreover, when a cast is needed to further restrain movement in the vicinity of the fractured bone, it is common practice to allow the pins to extend through the cast, which enables the surgeon, medical personnel, and/or the patient to readily inspect and/or clean the wound around the pins.

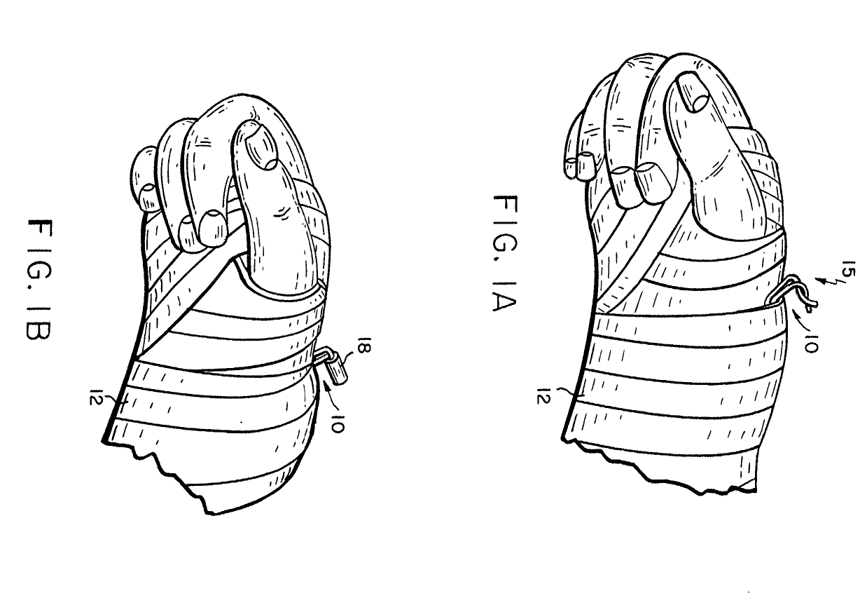

But for all the medical research that has gone into new and novel devices and methods to fixate fractured bones, very little has gone towards protecting the protruding pins used therefor. Indeed, as FIG. 1A illustrates, a common method of protecting the pin or pins 10 protruding through a post-operative dressing or cast 12 includes shunting the ends of the pins 10. Shunting typically involves twisting the free ends of a plurality of pins 10 into a resulting pigtail 15. Further, the pigtail 15 is often bent, or turned down, to minimize, or lower, its profile, so that the pigtail 15, having a plurality of sharp ends, is not sticking straight up where it is more prone to contact external objects, e.g., clothing, furniture, other people, and the like. Contact of the protruding pins with external objects can cause acute pain and discomfort to the patient and, if severe enough, can interrupt the bone healing process. In addition, the ends of the pins 10 can scratch and damage external objects and other persons as well.

FIG. 1B illustrates a second method of protecting the pin or pins 10 protruding through a post-operative dressing or cast 12, wherein the shunted pigtail 15 is encased in a, e.g., rubber or plastic, end cap 18. Although not shown in the figure, the capped pins 10 also can be turned down to minimize the profile of the capped pins 10. The utility of the end cap 18 is limited, however. Indeed, although the end cap 18 makes it more difficult for the sharp ends of the pins 10 to scratch or damage external objects, the end cap 18 does not prevent the capped pins 10 from catching on external objects. Hence, the end cap 18 fails to protect the pins 10 from transferring a load or force from an external source to the fractured bones, which, again, can cause acute pain and discomfort and/or interrupt the bone healing process.

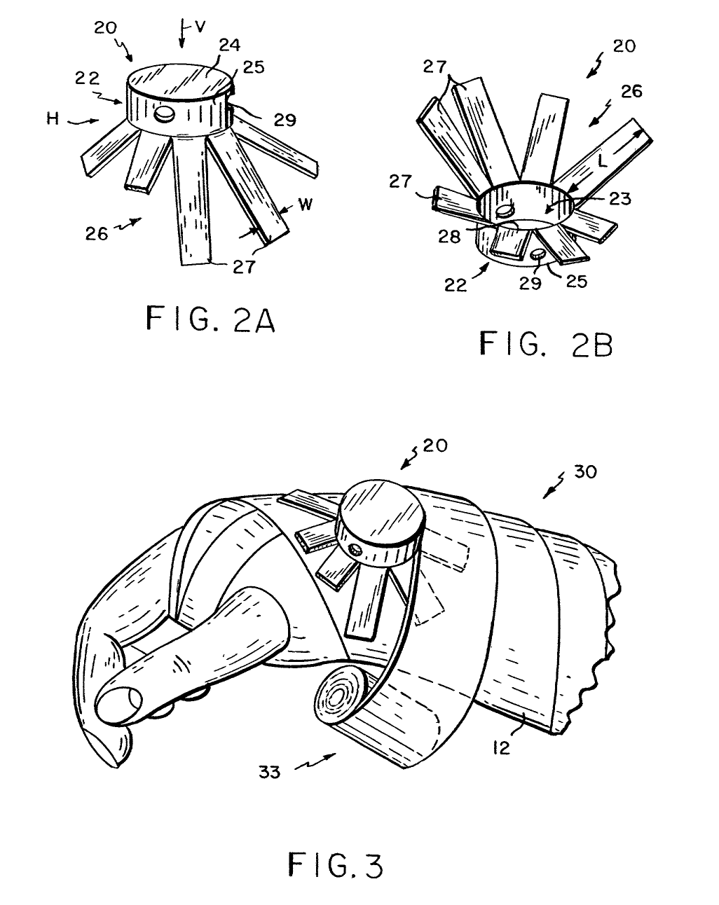

Referring to FIGS. 2A and 2B, an aspect of the present invention will be described. The embodied device 20 protects protruding orthopedic pins 10 from contact with external objects that might otherwise cause acute pain and discomfort and/or interrupt the bone healing process. Preferably, the device 20 comprises a rigid upper body 22, having an inner cavity 23, an upper surface 24, and an outer wall 25, and a conformable lower body 26, which, in a preferred embodiment, includes a plurality, and more preferably a multiplicity, of flexible legs or tabs 27.

For illustrative purposes only, the upper body 22 of the protective device 20 shown in FIGS. 2A and 2B has a generally cylindrical shape and the conformable lower body 26 of the protective device 20 includes eight substantially rectangular legs 27, thereby resembling an octopus. It should be noted, however, that the shapes of the upper and lower bodies 22 and 26 and the number of flexible legs 27 shown in FIGS. 2A and 2B are illustrative and are not to be taken or construed as limiting the practice of the invention thereto. Indeed, as further examples, the shape of the upper body 22 can be hemispherical, polyhedral, bulbous, and the like, while the shape of the legs 27 of the lower body 26 can be trapezoidal, polygonal, and the like.

In one aspect of the present invention, the protective device 20 is configured and arranged so that the inner cavity 23 of the upper body 22 completely encompasses and provides adequate room for one or more, e.g., shunted, orthopedic pins 10. Preferably, the protective device 20 is configured and arranged so that the orthopedic pins 10 do not contact the inner walls 21 or the underside 28 of the inner cavity 23. More preferably, the protective device 20 is configured and arranged so that the protective device 20 does not contact the orthopedic pins 10 when an external load or force, which is applied from any direction or axis, is applied to the device 20 even if the device 20 were distorted to a moderate extent by the multidirectional load or force. Indeed, when, for example, a vertical, or axial, external load or force V is applied to the device 20, the load or force V is borne initially by the upper surface 24, which then distributes the load or force V around the perimeter of the rigid upper body 22, which is to say, at a distance remote from the orthopedic pins 10.

In another aspect of the present invention, the lower body 26 of the protective device 20 is configured and arranged to be conformable and shapeable so that the plurality, or more preferably multiplicity, of flexible legs 27 can be disposed jointly or individually, e.g., substantially orthogonal to the outer wall 25, for the purpose of (i) attaching the device 20 to a surface surrounding the protruding pins 10, e.g., post-operative dressing or cast 12 or part of the human body, and (ii) providing a frictional surface area to resist horizontal, or radial, external loads or forces H acting on the device 20. Preferably, the flexible legs 27 of the conformable lower body 26 are configured and arranged such that the combined surface areas of the plurality of flexible legs 27 produce sufficient resisting force, e.g., through friction or adhesion, to prevent an external load or force H from displacing the device 20 sufficiently to cause it to contact the orthopedic pins 10. More preferably, the flexible legs 27 of the conformable lower body 26 are configured and arranged so that they can be securely and removably attached to a surface surrounding the pins 10, e.g., to a post-operative dressing or cast 12 or human body part, using an attaching device 33. Those skilled in the art can appreciate that the flexible legs 27 do not have to be of uniform length L and or width W to provide the necessary frictional resistance or adhesion. Indeed, as shown in FIG. 3, one aspect of the present invention includes flexible legs 27 of relatively longer and shorter lengths L, which make it easier to conform about the post-operative dressing or cast 12. Furthermore, preferably the flexible legs 27 can be trimmed easily, e.g., with scissors or a knife, to fit specific applications. In yet another embodiment, the lower body 26 of the device 20 can comprise a single, continuous flexible leg 27, giving the leg 27 the appearance of the brim of a hat. Preferably, however, a plurality of flexible legs 27 makes the device 20 more versatile as to where it can be applied.

In one aspect of the present invention, the upper body 22 and lower body 26 are fabricated, e.g., by at least one of extruding, molding, stamping, casting, and the like, as a single body, e.g., a cylinder, with the conformable lower body 26 being produced by fashioning a plurality, or more preferably a multiplicity, of flexible legs 27 therefrom, e.g., by cutting the cylinder in an axial direction. Preferably, the upper body 22 and/or lower body 26 is/are fabricated from at least one of plastics, including without limitation high density polyethylene (HDPE), polypropylene (PP), K-Resin® styrene-butadiene copolymers, polyethylene terephthalate ester (PETE), polyvinyl chloride (PVC), nylon, and polycarbonates, or from other materials including rubber, aluminum, pliable metals, dense paper stock, glass, and the like.

However, this is not to say that the upper and lower bodies 22 and 26 must be fabricated from the same material or that they be otherwise uniform. Indeed, in a separate aspect of the present invention, the upper body 22 and lower body 26 of the device are fabricated of different materials. For example, the upper body 22 can be fabricated from at least one of the above listed materials and the conformable lower body 26 can be fabricated from another of these materials or from at least one of Velcro®, cloth, elastic, leather, and the like, which are formed continuously with or fixedly attached to the upper body 22, e.g., by adhesives, stitches, rivets, staples, or snaps, by passing it through a slot or slit and attaching it on to itself, e.g., by gluing, stitching, riveting, stapling, snapping, and the like. In yet another embodiment, the upper and lower bodies 22 and 26 can be fabricated from a uniform material, but the lower body 26 can be made thinner than the upper body 22 to facilitate forming, trimming, and shaping the legs 27 and/or the lower body 26 can be scored, dimpled, coated with a non-skid material, provided with an adhesive, and the like to produce additional frictional resistance and adhesion.

In another aspect of the device 20 of the present invention, one or more air holes 29 are disposed through the outer wall 25 to the inner cavity 23 to allow fresh air to circulate in and around the wound. Alternately, the device 20 can be fabricated from a “breathable” material, e.g., a non-woven plastic, through which fresh air can easily circulate.

An aspect of a system 30 and method for protecting surgical pins 10 will now be described using FIG. 3 as a reference. Preferably, the system 30 for protecting surgical pins 10 includes the device 20 described above in combination with an attaching device 33. More preferably, the system 30 for protecting surgical pins 10 includes the device 20 described above in combination with at least one of self-adherent tape, elastic bandages, elastic tapes, surgical tapes, gauze, and the like. In application of this system 30, the upper body 22 of the device 20 is configured and arranged, e.g., centered, about the protruding pins 10 and the flexible legs 27 of the lower body 26 are configured and arranged about the post-operative dressing or cast 12, e.g., in a radial pattern. The attaching device 33 can then be configured and arranged over the extended legs 27 of the lower body 26, e.g., by wrapping, such that the flexible legs 27 are held tightly to the post-operative dressing or cast 12, and, moreover, the upper body 22 is held firmly in place about the protruding pins 10. When necessary, the flexible legs 27 can be temporarily attached to the area surrounding the protruding pins 10 with, e.g., a strip of surgical tape, to hold the lower body 26 in place while the attaching device 33 is wrapped around the flexible legs 27. The end of the attaching device 33 can be tucked under one of the wrappings and/or removably attached or fastened onto itself, the post-operative dressing or the cast 12 using at least one of the self-adhering properties of the attaching device 33 and/or a separate fastener, clasp, surgical tape, and the like. The best mode of practicing this invention, however, includes self-adherent tape as an attaching device 33.

In another aspect of the attaching device 33, the attaching device 33 comprises a wristband-like structure (not shown) from which an opening is removed, which opening corresponds in approximate dimension to that of the upper body 22 of the device 20. Accordingly, the wristband-like attaching device is configured and arranged so that the opening passes over the upper body 22 of the device 20 but not the flexible legs 27 of the conformable lower body 26. Preferably the wristband-like structure can wrap around the surface surrounding the pins 10, e.g., the post-operative dressing or cast 12, and either fasten onto itself using, e.g., a Velcro® means, a belt and buckle means, a lacing means, and the like, or be a closed loop that is held in place elastically, e.g., resembling and being worn like an athletic sweat band. Alternately, in a separate embodiment, the conformable lower body 26 of the device 20 comprises a wristband-like structure that is fixedly attached to the upper body 22 by at least one of gluing, stitching, stapling, snapping, and the like.

In a separate embodiment, the flexible legs 27 of the conformable lower body 26 comprise a plurality of mating Velcrot strips, which are, e.g., glued, stitched, riveted, stapled, snapped, and the like to the upper body 22 of the device 20. The Velcro® strip legs 27 can be configured and arranged around the surface surrounding the protruding pins 10 and mated and/or can be removably attached, e.g., to the gauze-like surface of the post-operative dressing or cast 12 and/or to corresponding Velcro® strips that are attached to the outer surface of the post-operative dressing or cast 12 for that purpose. In yet another embodiment, the flexible legs 27 of the conformable lower body 26 can include an adhesive on their underside that can removably and adhesively attach each leg 27 to, e.g., the post-operative dressing or cast 12 and/or an adhesive can be applied to an area of the post-operative dressing or cast 12 surrounding the protruding pins 10.

While a number of embodiments of the invention have been described, it should be obvious to those of ordinary skill in the art that other embodiments to and/or modifications, combinations, and substitutions of the present invention are possible, all of which are within the scope and spirit of the disclosed invention.

For example, the upper body 22 of the device can take on a fanciful or decorative shape. Indeed, the shape of the upper body 22 can be based on the shape of, e.g., a baseball, golf ball, football, frog, favorite pet, a cartoon character, historical figure, and the like or, in a separate embodiment, the upper body 22 can be configured and arranged to connect to, e.g., by tight interference fit, a plurality of removable attachments having the shape of, e.g., a baseball, golf ball, football, frog, favorite pet, cartoon character, historical figure, and the like. This embodiment can include the decorative shape being applied as an over-cap that is disposed over and removably attached to the upper body 22 of a device 20, which is fabricated of a transparent material to allow inspection of the wound and/or pins without having to remove the device 20.

In another embodiment, the upper body 22 and lower body 26 comprise separate, non-contiguous elements of the device 20 so that the entire device 20 does not have to be removed to inspect the wound and/or pins 10. For example, in this embodiment, the upper body 22 can be configured and arranged to be slightly larger in dimension than the conformable lower body 26. Accordingly, the lower body 26 can be removably attached to, e.g., the postoperative dressing or cast 12 in any manner previously described herein and a portion of the upper body 22 fits over a portion of the lower body 26 to form a tight interference fit. Similarly, the upper and lower bodies 22 and 26 can be threaded such that the upper body 22 screws onto the lower body 26.

In yet another embodiment, the upper body 22 can be configured and arranged to be slightly smaller in dimension than the lower body 26. Accordingly, the upper body 22 can be removably inserted into the lower body 26 to form a tight interference fit. Similarly, the upper and lower bodies 22 and 26 can be threaded such that the upper body 22 screws into the lower body 26.

In still a further embodiment, when the upper and lower bodies 22 and 26 comprise two discrete elements that can be removably attached to one another, as described above, the lower body 26 can be integrally attached to the cast 12 by means that are well known to the art. Also, when the upper and lower bodies 22 and 26 comprise two discrete elements that can be removably attached to one another, for the convenience of the patient the upper and lower bodies 22 and 26 can be tethered to one another by means that are well known to the art.

Furthermore, while the description herein may have implied that the orthopedic pins 10 are protruding through a post-operative dressing or cast 12, the invention can also be practiced without a post-operative dressing or cast 12, whereby the upper body 22 of the device 20 is configured and arranged about the protruding pins 10 and the legs 27 of the lower body 26 are configured and arranged on a part of the human body; the attaching device 33 being removably attached to the body part in a manner described above.

Moreover, the description herein may have implied that the invention is useful only to human beings. However, in another embodiment, the device 20 can be used to protect protruding pins 10 used to set the fractured bones of animals as well as humans.

Similarly, the type of wound to be protected, whether on man or beast, does not have to be protruding pins 10 and can include practically any surficial, external wound. In this case, the device 20 provides a means to protect a wound, e.g., bums, surgical sutures, and the like, from external contact without the necessity of any dressing being in contact with the wound area.

What is claimed is:

1. A device for protecting at least one of one or more surgical pins that protrude through at least one of a patient's skin, a post-operative dressing, and a cast and a wound, the device comprising:
a rigid upper body, having a shape, which shape includes an inner cavity, an upper surface, and an outer wall, for encompassing and protecting one or more of said surgical pins; and
a conformable lower body for removably attaching the device to a surface surrounding said one or more surgical pins.

2. The device as recited in claim 1, wherein the device further includes one or more ventilation holes in the outer wall of the rigid upper body to allow air to enter and circulate in the inner cavity.

3. The device as recited in claim 1, wherein the shape of the rigid upper body is at least one of cylindrical, hemispherical, polyhedral, bulbous and fanciful, including a golf ball, a baseball, a football, an animal, a favorite pet, a cartoon character, and a historical figure.

4. The device as recited in claim 3, wherein the shape of the rigid upper body is substantially cylindrical.

5. The device as recited in claim 1, wherein the conformable lower body comprises a plurality of flexible legs.

6. The device as recited in claim 5, wherein, said plurality of flexible legs is integral to and contiguous with said rigid upper body.

7. The device as recited in claim 5, wherein each of said plurality of flexible legs has a length and a width that are adjustable.

8. The device as recited in claim 1, wherein the conformable lower body is integral to and contiguous with the rigid upper body.

9. The device as recited in claim 1, wherein the rigid upper body and conformable lower body are formed from similar materials comprising at least one of plastic, rubber, aluminum, pliable metals, glass, and dense paper stock.

10. The device as recited in claim 1, wherein the rigid upper body and conformable lower body are formed from dissimilar materials.

11. The device as recited in claim 10, wherein the conformable lower body is fixedly attached to the rigid upper body by at least one of gluing, stitching, riveting, stapling, snapping, and passing an end of the conformable lower body through a slit provided therefor in the rigid upper body and attaching said end onto said conformable lower body.

12. The device as recited in claim 1, wherein the device further includes one or more over-caps that are removably attachable to the rigid upper body.

13. The device as recited in claim 12, wherein the one or more over-caps have a shape, the shape comprising at least one of a golf ball, a baseball, a football, an animal, a favorite pet, a cartoon character, and a historical figure.

14. The device as recited in claim 1, wherein the conformable lower body comprises at least one of Velcro®, cloth, leather, and elastic.

15. A system for protecting one or more surgical pins that protrude through at least one of a patient's skin, a post-operative dressing, and a cast, the system comprising:
a protecting device for encompassing said surgical pins, the protecting device further comprising:
a rigid upper body having a shape, which shape includes an inner cavity, an upper surface, and an outer wall; and
a conformable lower body; and
an attaching device for removably attaching said protecting device to a surface surrounding the one or more surgical pins.

16. The system as recited in claim 15, wherein the attaching device comprises at least one of a self-adherent tape, elastic bandage, elastic tape, surgical tape, and gauze.

17. The system as recited in claim 15, wherein the attaching device comprises an adhesive that is disposed between the surface surrounding the one or more surgical pins and the attaching device.

18. The system as recited in claim 15, wherein the attaching device comprises a flexible wristband having an opening, wherein said opening is sufficiently large so as to slip over the rigid upper body and flexibly restrain the conformable lower body.

19. The system as recited in claim 15, wherein the rigid upper body is removably attachable to the conformable lower body, which conformable lower body is integral to at least one of the post-operative dressing and the cast.

20. A method of protecting one or more surgical pins from contact with an external object, the method comprising the steps of:
encompassing said one or more surgical pins in a rigid body, wherein said rigid body is configured and arranged such that it does not contact said one or more surgical pins when an external force is applied to said rigid body;
removably attaching the rigid body to a surface surrounding said one or more surgical pins using an attaching device.

21. The method as recited in claim 20, wherein the method further comprises the step of:
ventilating said one or more surgically pins by providing at least one ventilation hole in the rigid body.

22. The method as recited in claim 20, wherein the rigid body is removable attached to the surface surrounding the one or more surgical pins by wrapping a plurality of flexible legs that are fixedly attached to said rigid body with at least one of a self-adherent tape, elastic bandage, elastic tape, surgical tape, and gauze.
